(19) United States
(12) Patent Application Publication
(10) Pub. No.: US 2006/0177882 A1
(43) Pub. Date: Aug. 10, 2006

(54) Immunoassays with enhanced selectivity

(76) Inventors: Samad Talebpour, Richmond Hill (CA); Stephen W. Leonard, Unionville (CA); Pasha Emadi-Konjin, Richmond Hill (CA)

Correspondence Address: Ralph A. Dowell of DOWELL & DOWELL P.C., 2111 Eisenhower Ave, Suite 406, Alexandria, VA, 22314, US

(21) Appl. No.: 11/050,443

(22) Filed: Feb. 4, 2005

(51) Int. Cl. G01N 33/537; G01N 33/558

(52) U.S. Cl. 435/7.92; 436/514

(57) ABSTRACT

The present invention provides a method for the detection of an analyte in a specimen comprising two steps. First, the specimen is contacted with a substrate having receptors bound thereon, having higher affinity for cross-reactive substances than for the analyte, in order to increase the ratio of the analyte to the cross-reactive substances. Then, the treated specimen is assayed by using another receptor while decreasing the effects of the cross-reactive substance contained in the original specimen. The receptors in the first step are preferably molecular imprinted polymers that are capable of absorbing multiple cross-reactive substances.

### FIELD OF THE INVENTION

The present invention is related to immunoassays with enhanced selectivity for detecting the presence or absence of a target analyte which uses a molecularly imprinted polymer (MIP) having low affinity to the target analyte, but high affinity for one or more cross-reactive substances.

### BACKGROUND OF THE INVENTION

Affinity-based immunoassays, due to their sensitivity, are routinely used to detect and measure the presence and the concentration of an analyte in a sample. The analyte may be any of the wide variety of materials, such as drugs, pollutants, chemicals, contaminants, or the like. These assays need high-affinity specific receptors, but obtaining a very specific receptor that will only bind to one specific analyte is not an easy task because there are generally several analogues and/or metabolites of the target analyte, which we will call cross-reactive substances.

An established method to decrease the deteriorating effects of cross-reactive substances is to employ a two-site binding assay. In this case, two receptors are used which selectively recognize two independent sites of a target molecule. Unfortunately, this method is not applicable when the analyte has a low molecular mass (<1000). In these cases, competitive immunoassays represent the most common approach for performing detection and quantification. These immunoassays, whatever their format, contain a target analyte, a specific antibody that binds to the analyte and a corresponding labeled-analyte, which consists of a derivative of the target molecule attached to a detectable label such as an enzyme. The principal strategy for improving the assay selectivity has been oriented at improving the specificity of the detecting antibody by judicious choice of hapten used to raise antibodies. Another strategy, suggested in U.S. Pat. No. 6,306,616, is based on neutralizing the effect of the interfering substance in the sample. This technique, in principle is used to confirm the positive assay, and thus is not intended for quantitative measurement of the analyte.

An interesting possibility as a sorbent medium for the removal of cross-reactants from an assay involves the use of molecularly imprinted polymers (MIPs). The preparation of an MIP is based on the principle of utilizing the functionality of a target molecule (the template), in order to assemble its own recognition site by forming interactions with complementary functional groups of appropriate functional monomers. These interactions are then frozen by polymerization, carried out in a solution with a high concentration of cross-linker. Subsequent removal of the template by extraction creates binding sites, ideally maintaining the precise spatial arrangement of the functional groups. Thus, reversible re-binding and highly selective recognition of the target molecule is achievable, and it is possible to perform MIP-based assays.

Such materials, owing to their specificity, short development time, ease of preparation, low price and high chemical and physical stability, have also been considered as a viable alternative to antibodies for use as receptors in immunoassays. In general, however, the reduced affinity relative, inhomogeneity of binding sites, and lack of reproducibility make MIPs better suited to applications involving separation and extraction of substances rather than complete assays. Recently, it has been shown by Spegel (P. Spegel et al., Anal Chem 75 (2003) 6608-6613) that it is possible to template an MIP for multiple analytes. Although such a multiple-templated MIP was discussed in the limited context of a recognition element for the separation and detection of multiple target analytes, it is also possible to use it as a means of absorbing multiple cross-reactants in order to obtain an assay with enhanced specificity for a single analyte, as described in the present invention.

A novel usage of MIPs as sorbent media for the removal of cross reactants has been described in U.S. Pat. No. 6,461,873, in which a device using an MIP to enhance the selectivity of caffeine detection is disclosed. The device is a chromatographic caffeine detector that uses the finite absorption of a MIP templated for caffeine as a chromatographic medium, where samples with higher concentrations of caffeine migrate further up the chromatographic medium. Chromogenic reagents are used to enable a colourimetric measurement of the caffeine migration distance, which can be directly related to the caffeine concentration. The device also includes a sorbent zone, in which the sample is passed through a strip of MIP that absorbs cross-reactive substances that interfere with the quantification of caffeine. This usage of the MIP sorbent zone increases the specificity and detection limit of the subsequent chromatographic assay. Although this device appears to perform well as a detector for caffeine, it is limited in the scope of its physical nature and assay format to strip-based chromatographic assays.

What is therefore needed is a method of performing an MIP-based sorbent assay for non-chromatographic binding assays, especially those in which multiple cross-reactants are present.

### SUMMARY OF THE INVENTION

The disadvantages of immunoassays resulting from the cross-reactivity of receptors are overcome by using the methods of this invention. In the preferred embodiment, a sample is assayed in two steps. In the first step, a liquid sample is brought in contact with a molecularly imprinted polymer (MIP) having low affinity to the target analyte, but high affinity for one or more cross-reactive substances. Thus, the concentrations of the cross-reacting species in the liquid sample are reduced. In the second step, the liquid sample is separated from the MIP and assayed with a binding assay known in the prior art to determine the presence or absence of the target analyte. If the separated liquid sample is aliquoted into several separate vessels, then multiple binding assays for different target analytes can be performed. The MIP employed in the invention can be either cast for a single cross-reactant or cast for multiple cross-reactants that may be present in the sample. A multi-receptor MIP absorbs multiple interfering cross-reactants and dramatically increases the specificity of the subsequent assay.

Thus, in one aspect the present invention provides a method of performing a binding assay for one or more target analytes, comprising the steps of:
a) contacting a liquid sample that may contain one or more target analytes and one or more cross-reactants with a molecularly imprinted polymer (MIP) that is templated to absorb one or more of said cross-reactants;
b) incubating said liquid sample with said MIP for a time interval;
c) separating said liquid sample from said MIP; and
d) performing one or more binding assays on said separated liquid sample for one or more target analytes.

According to another aspect of the invention, an MIP cast for one or more cross-reactants is incorporated into a lateral flow assay binding assay device. The device includes a section of a porous strip that incorporates an MIP cast for one or more cross-reactants. As the sample and reagents flow through the layer of the porous strip containing the MIP, cross-reactants are absorbed and the specificity of the assay is enhanced. The subsequent binding of analyte and reagents in the capture zone of the lateral flow porous strip produces a result that has an increased specificity over a prior-art device lacking the MIP section.

Thus, in another aspect of the invention there is provided a lateral flow binding assay device comprising:
a) a sample addition pad that is in direct contact with a proximal end of a porous strip;
b) a first zone within said porous strip that is adjacent to said proximal end of said porous strip and contains an MIP that is templated to absorb one or more of said cross-reactants;
c) a second zone located beyond said first zone within said porous strip comprising one or more localized regions of analyte or receptors bound to said strip, whereby the presence of an analyte can be visually determined following a binding reaction between an analyte within said sample and a reagent; and
d) an absorbing material located at a distal end of said porous strip.

A further understanding of the functional and advantageous aspects of the invention can be realized by reference to the following detailed description and drawings.

### BRIEF DESCRIPTION OF THE DRAWINGS

The invention will be more fully understood from the following detailed description thereof taken in connection with the accompanying drawings, which form a part of this application, and in which:

FIG. 1 is a block diagram showing the steps in which the immunoassay is performed in accordance with the method of the present invention;

FIG. 3 shows plots of the normalized signal in a competitive assay as a function of the analyte concentration for a single-step immunoassay in the presence of a cross-reactant. The initial concentration of the cross-reactant is chosen as a parameter;

FIG. 3 shows plots of the ratio of the analyte and the cross-reactant concentrations for treated and untreated samples;

FIG. 4 shows plots of the normalized signal in a competitive assay as a function of the analyte concentration, for a two-step immunoassay in the presence of a cross-reactant where a multi-cast MIP is used to pre-treat the sample in which the initial concentration of the cross-reactant is chosen as a parameter; and

FIG. 5 is a perspective view of a lateral flow device for use in the assay of the present invention.

### DETAILED DESCRIPTION OF THE INVENTION

The assay method of the present invention employs a molecularly imprinted polymer (MIP) to selectively absorb cross-reactants that would otherwise reduce the specificity of an assay for a target analyte. More specifically, a novel method of conducting an assay is disclosed wherein a sample that may contain a target analyte and also cross-reactants that may interfere with the signal measured to infer the concentration of the target analyte is first contacted with an MIP solid phase that selectively absorbs the cross-reactants. After the sorbent step of the inventive assay, a binding assay known in the prior art may be used to detect the concentration of target analyte in the sample. The removal of cross-reactants via the MIP dramatically increases the specificity of the overall assay.

The term “molecularly imprinted polymer (MIP)”, as used herein, means an artificial receptor for an analyte made by forming recognition sites within a polymer matrix that are adapted to the three-dimensional structure of the analyte molecule, that is, it is templated to absorb one or more of pre-selected analytes (or cross-reactants as in the case of the present invention). MIPs can be engineered via covalent and non-covalent interactions and metal ion coordination. Their synthesis generally involves the interaction of a functional monomer with the analyte, following which the monomer-analyte complex is polymerized via a cross-linker, producing a macroporous polymer with the analyte molecules in a sterically fixed arrangement. Artificial recognition sites for the analyte are obtained after the removal of the analyte from the MIP. Although the traditional recipe for the syntesis of MIPs involves the use of methyl methacrylate (MMA) as a functional monomer and ethylene glycol methacrylate (EGDMA) as the cross-linker, a wide variety of methods and formats are known in the prior art may be used which fall within the scope of the present invention.

The term “receptor”, as used herein, means antibodies, DNA, RNA, aptamers, molecularly imprinted polymers, or any other species capable of exhibiting a specific binding affinity for the analyte.

The phrase “analyte” or “target analyte”, as used herein, means any species whose presence or concentration in a sample is sought.

The phrase “cross-reactant”, as used herein, means any species whose presence or concentration in a sample produces a signal that can be misinterpreted as having been generated by the presence of analyte.

The phrase “binding assay”, as used herein, means an assay in which the affinity of a receptor for a target analyte is employed to bind the receptor to the analyte a produce a measurable signal that is proportional to the analyte concentration.

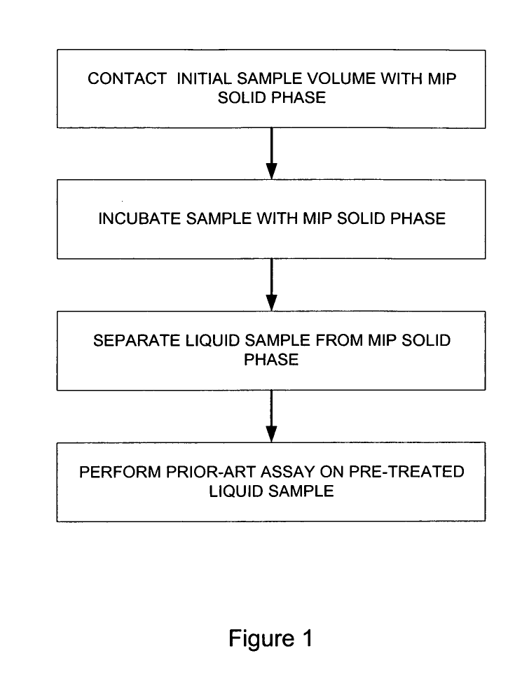

In one embodiment of the invention, an immunoassay is employed as schematically illustrated in FIG. 1. In the first step, a liquid sample which may contain one or more target analytes and one or more cross-reactants is contacted with a MIP solid phase on which there are receptors for the cross reactant. The receptors are characterized by a high affinity for the cross-reactant(s) but not for the target analyte(s). After incubating the sample with the MIP solid phase for a prescribed time interval, the concentration of the cross-reactive substance decreases. After the completion of the incubating step, the liquid sample is separated from the MIP solid phase and a binding assay is used to detect the presence of the analyte. The method therefore increases the effective specificity of the assay by the ratio of the analyte concentration to the cross-reactant concentration after pre-treatment.

An example of such a binding assay is a homogeneous, non-separation enzyme based competitive assay. Another example is a two-site binding enzyme-linked immunosorbent assay (ELISA). Since the first step does not alter the composition of the specimen beyond the removal of cross-reactants, the treated specimen can be used in virtually any assay platform for the analyte.

In order to have the maximum possible surface area, it is preferable to prepare MIPs as monosized spherical particles rather than as monoliths that must be fragmented into irregular particles. An example of a prior-art method to achieve this is U.S. Pat. No. 5,872,198, in which suspension polymerization in perfluorocarbon liquids was employed, which patent is incorporated herein in its entirety. Alternatively, magnetic beads coated with an MIP can be used in the sorbent vessel for the initial absorption step. Such beads can be prepared by techniques provided in the prior art, e.g., U.S. Pat. No. 6,316,235, which patent is incorporated herein in its entirety. The separation of the treated liquid sample from the MIP solid phase can then be achieved using a magnetic field.

Without being bound by any theory, the preceding description of the inventive assay method is henceforth described via a specific non-limiting mathematical example. The immunoassay involves one type of labeled-analyte (Ag^{*}), with initial concentration of C_{c0}, is competing with analyte in the solution (Ag), with initial concentration of Cₐ₀, and a single type of cross-reactive substance (Ag^{r}), with initial concentration of Cᵣ₀, to bind to antibodies (Ab) coated with an average surface density Σ on a solid surface with total surface area S. The three reactions occurring in the liquid and solid phases are described with the following equilibrium equations (see reference [2])
Cₐ·σ=Kₐσₐ, (1)
Cᵣ·σ=Kᵣσᵣ, (2)
C_{c}·σ=K_{c}σ_{c}, (3)
where σ is the surface density of un-reacted Ab, σₐ, σᵣ and σ_{c} are the surface densities of AgAb, Ag^{*}Ab and Ag^{c}Ab, and Cₐ, C_{c} and Cᵣ are the equilibrium concentrations of Ag, Ag^{*} and Ag^{r}, respectively. The law of the conservation of reagents results in
Cₐ+fσₐ=Cₐ₀, (5)
Cᵣ+fσᵣ=Cᵣ₀, (6)
C_{c}+fσ_{c}=C_{c0}, (7)
σ+σₐ+σᵣ+σ_{c}=Σ, (8)
where f≡S/V, and V is the reaction volume of the immunoassay. In FIG. 2, the normalized signal σ_{c}/σ_{c,max} is shown as a function of Cₐ₀, with Cᵣ₀ as a parameter. The parameters have been chosen as Kₐ=1 nM, K_{c}=1 nM, Kᵣ=20 nM, f=0.5/2×10⁻⁴=2500 cm²/L and Σ=6.0×10⁻⁶ nM/cm². As it is observed the signal strongly depends on Cᵣ₀. Any variation in the concentration of the cross-reactant therefore translates directly into strong variations in the signal amplitude, particularly over the range where the analyte concentration is low. The coefficient of variation of such an assay, when measured over a pool of samples with variations in the amount of cross-reactant, will therefore be high and likely unacceptable over a wide region of the desired dynamic range.

Now consider a case that the sample is pretreated by incubating it with a MIP surface, with surface area S₁, coated with receptors, R, with density of Σ₁. The volume of the incubation well is V₁. The two reactions occurring in the liquid and solid phases are described with the following equilibrium equations
Cₐ₁·σ₁=Kₐ₁σₐ₁, (9)
Cᵣ₁·σ₁=Kᵣ₁σᵣ₁, (10)
where σ₁ is the surface density of un-reacted receptors, σₐ₁ and σᵣ₁ are the surface density of AgR, and Ag^{r}R, Cₐ and Cᵣ (mol/l) are the equilibrium concentrations of Ag and Ag^{r}, respectively. The law of the conservation of reagents results in
Cₐ₁+f₁σₐ₁=Cₐ₀, (11)
Cᵣ₁+fσᵣ₁=Cᵣ₀, (12)
σ+σₐ₁+σᵣ₁=Σ₁. (13)

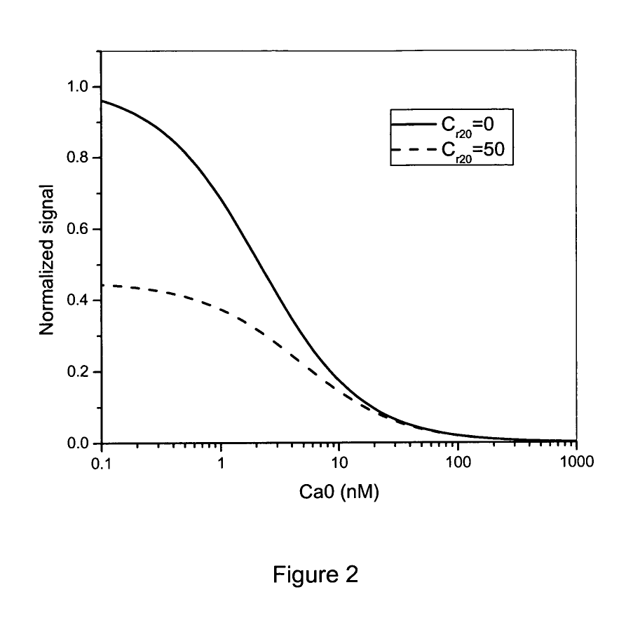

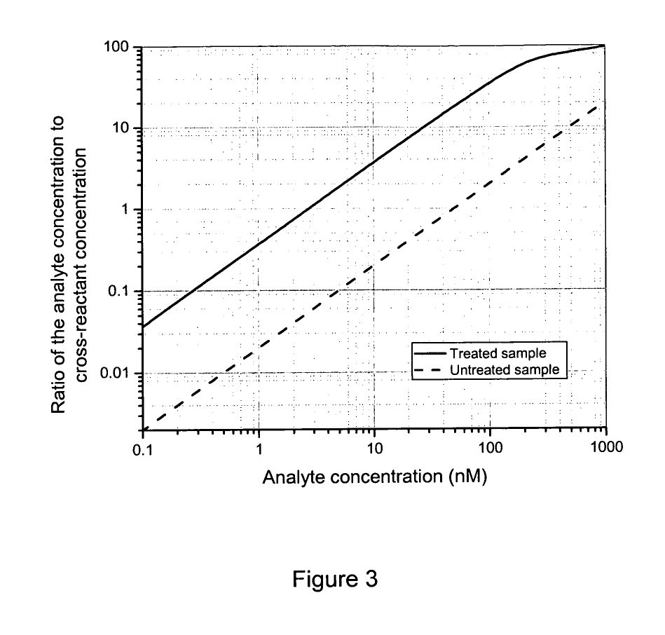

Solving these equations demonstrates that as a result of the pretreatment, the concentration of both analyte and the cross-reactive substance decrease, although with different magnitudes. For the values Kₐ₁=20 nM, Kᵣ₁=1 nM, f₁=5/0.2×10⁻⁴=2.5×10⁵ cm²/L and Σ=3.0×10⁻³ nM/cm², we have presented the value of Cₐ₁/Cᵣ₁ as a function of Cₐ₀ in FIG. 3. On the same Figure we have included Cₐ₀/Cᵣ₀, i.e., the ratio of the concentrations in the untreated sample. As can be seen from FIG. 3, the pretreatment of the sample with the MIP surface increases the analyte to cross-reactant ratio by a factor of twenty. This dramatic increase in the analyte to cross-reactant ratio translates directly into a twenty times increase in the assay specificity.

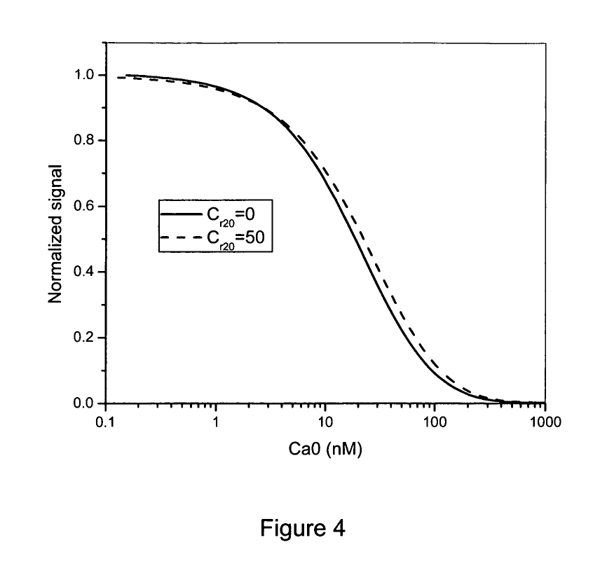

FIG. 4 shows the dose-response curve of the assay described above. The solid line shows the assay performance in the absence of cross-reactants. When cross-reactants are present with a high concentration, however, the pretreatment step with the MIP ensures that there is little effect on the assay, as shown by the dashed line. This immunity to cross-reactants, when compared to FIG. 2, reveals the superiority of the MIP assay scheme over prior art methods.

Although the above method has illustrated the use of an MIP templated for a single cross-reactant, a preferred embodiment of the invention uses multiple MIPs to selectively absorb multiple cross-reactants. One method of producing an MIP that can absorb multiple cross-reactants is to mix several MIPs that are separately made for single cross-reactants. Prior-art methods for making MIPs with multiple solid phases, including MIP nanoparticles and MIP-coated magnetic beads, are preferably used for producing singly-templated MIPs that can be combined to form the desired mixed MIP.

Another approach for producing an MIP that can absorb multiple cross-reactants is to create an MIP that is templated with multiple cross-reactants in a single step. Such MIPs, henceforth referred to as multi-receptor MIPs have been studied in the academic literature and methods for their synthesis are known (P. Spegel et al., Anal Chem 75 (2003)6608-6613). The formation of a suitable solid surface for use as an absorber can be achieved using any of several prior-art methods, including the formation of MIP nanoparticles and the synthesis of MIP magnetic beads. In another preferred embodiment of the invention, the multi-receptor MIP is coated onto the surface of a vessel, for example the bottom of a microplate well or the inner surface of a pipette tip.

The preceding discussion clearly illustrates the dramatic performance enhancement obtained by pre-treating the sample with a MIP that absorbs one or more potential cross-reactants. The removal of cross-reactants in a systematic and controlled manner surmounts a key limitation in binding assay technology: non-specificity. This drawback has limited the use of binding assays, in particular immunoassays, to the realm of qualitative or semi-quantitative assays due to their propensity for generating false-positive results. Indeed, immunoassays are often simply used to screen out negative results, in which case any positive results are sent for expensive and time-consuming confirmatory testing. With the removal of multiple known cross-reactants from the sample prior to conducting the assay, the multiple MIP approach enables confirmatory-type immunoassays in a simple and inexpensive platform.

In addition to enabling confirmatory-type immunoassays, the invention also provides a means of using sub-optimal antibodies for screening assays. In many cases, it is not feasible to generate antibodies with high affinity and/or specificity for a given analyte. This may be due to a multitude of factors that result in inferior antibodies, including difficulties in generating suitable derivatives of small molecule analytes in order to generate in immunogenic response and constraints on development time. The multiple MIP scheme disclosed herein allows the use of such inferior antibodies, since the pretreatment step in which the multiple MIP absorbs cross-reactants effectively multiplies the specificity of the subsequent assay by a potentially large factor.

In another embodiment, the sample is introduced into a column that is packed with a multiple MIP. The sample flows through the column (pressure may be applied via a pumping mechanism) and the MIP absorbs the cross-reactants owing to the very high surface area inside the column. After the sample flows through the column, it is collected into an intermediate vessel, from where it is accurately pipetted into a reaction vessel. In this scheme, the sorbent column can either be made disposable or eluted and washed for reuse.

Although the assay is preferably carried out by first contacting the sample with the MIP and subsequently performing the assay with a purified form of the sample, the assay may also be conducted simultaneously with the MIP in a non-separation format. However, this scheme is usually precluded by the need to have a high MIP surface area to liquid volume ratio. If this ratio is not sufficiently high, only a modest fraction of the cross-reactants will be absorbed by the MIP and the assay specificity will not be dramatically enhanced.

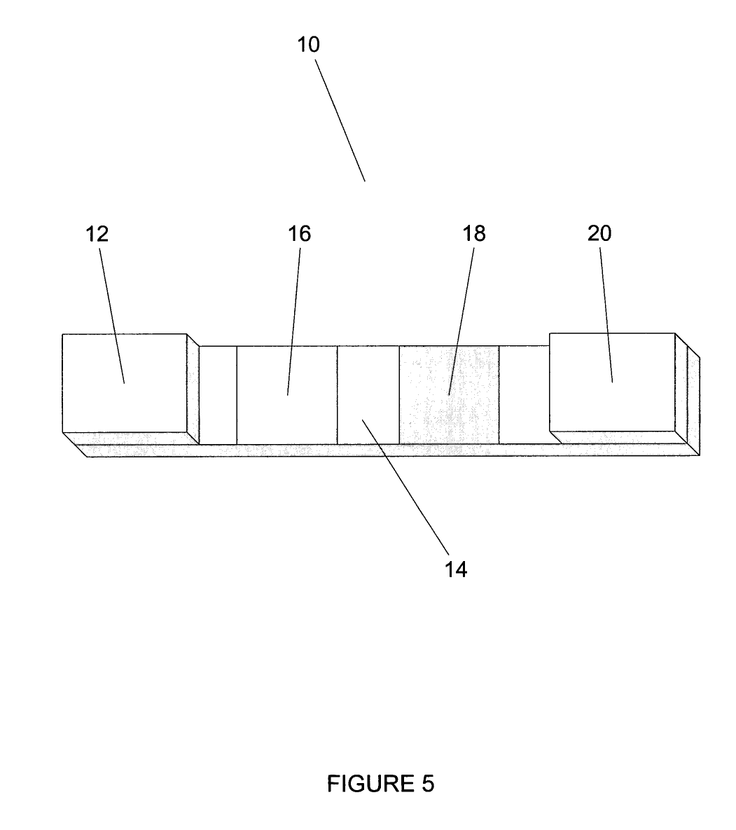

An MIP can also be used as a sorbent material in a lateral flow binding assay in order to increase the assay specificity. Referring to FIG. 5, the lateral flow device 10 includes a sample reservoir 12 that is placed in contact with a porous strip 14. Dried reagents are incorporated into the sample reservoir 12, eliminating the need for the addition of separate liquid reagents. The dried reagents are present for the purpose of generating a signal through a binding reaction with either the analyte or a receptor designed to bind with a reagent immobilized in the capture zone downstream of the reservoir 12. A non-limiting example of a reagent may be colloidal gold particles with receptors bound thereto for binding with the analyte. Gold particles labeled with analyte itself may be used as the reagent in the sample reservoir 12 in some embodiments. Fluorescent species may also be used in various configurations as dried reagents in reservoir 12. An MIP cast for one or more cross-reactants is incorporated into the nitrocellulose membrane in the zone 16 adjacent to the sample reservoir 12.

As the sample and solubilized reagents percolate through this MIP zone 16 via capillary action, cross-reactants are absorbed. After passing through the MIP zone 16, the sample and solubilized reagents encounter the capture zone 18. The immobilization of analyte or reagents within the capture zone produces a visual measure of the analyte concentration. A control line may be integrated in the capture zone to enhance the accuracy of the assay result. An absorption pad 20 located beyond the capture zone absorbs sample and reagents as they pass through the capture zone, eliminating backflow. The absorption of interfering cross-reactants by the MIP dramatically decreases the incidence of false-positive assay results.

In another embodiment of the invention, the sample first passes through the MIP zone 16 before encountering an additional zone containing one or more dried reagents, where this additional zone is located between the MIP zone 16 and the capture zone 18. In this format, the MIP advantageously absorbs cross-reactants that may be present before the sample solubilizes and possibly reacts with the dried reagents. The pre-absorption of cross-reactants thus reduces or eliminates the potential of cross-reactants to react with the reagents and produce an erroneous assay result.

It will be apparent to those skilled in the art that there are many possible forms of the invention that, while differing from the aforementioned embodiments, do not depart from the scope and theme of the invention. For example, the binding agent in the final assay may also be a MIP, or alternatively an aptamer, instead of an antibody.

As used herein, the terms “comprises”, “comprising”, “including” and “includes” are to be construed as being inclusive and open ended, and not exclusive. Specifically, when used in this specification including claims, the terms “comprises”, “comprising”, “including” and “includes” and variations thereof mean the specified features, steps or components are included. These terms are not to be interpreted to exclude the presence of other features, steps or components.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

### REFERENCES

[1] P. Spegel et al., Anal Chem 75 (2003) 6608-6613.
[2] C. Domenici et al., Biosensors & Bioelectronics 10 (1995) 371-378

Therefore what is claimed is:

1. A method of performing a binding assay for one or more target analytes, comprising the steps of:
a) contacting a liquid sample that may contain one or more target analytes and one or more cross-reactants with a molecularly imprinted polymer (MIP) that is templated to absorb one or more of said cross-reactants;
b) incubating said liquid sample with said molecularly imprinted polymer for a time interval;
c) separating said liquid sample from said molecularly imprinted polymer; and
d) performing one or more binding assays on said separated liquid sample for one or more target analytes.

2. The method according to claim 1 wherein said molecularly imprinted polymer is coated onto the inner surface of a vessel.

3. The method according to claim 2 wherein said molecularly imprinted polymer is coated onto the bottom of a well in a microtitre plate.

4. The method according to claim 2 wherein said liquid sample is separated from said molecularly imprinted polymer by aspiration of said liquid sample from said vessel.

5. The method according to claim 2 wherein said molecularly imprinted polymer is coated onto the inner surface of a pipette tip.

6. The method according to claim 1 wherein said molecularly imprinted polymer is a collection of MIP-coated magnetic particles that are separated from said liquid sample via a magnetic field.

7. The method according to claim 1 wherein said molecularly imprinted polymer is made from a mixture of several individual molecularly imprinted polymers that are each individually cast for a separate cross-reactant prior to being mixed together, the resulting mixture of several individual molecularly imprinted polymers being capable of absorbing multiple cross-reactants.

8. The method according to claim 1 wherein said molecularly imprinted polymer is a multi-receptor MIP in which casts for multiple analytes are incorporated into the MIP, the resulting MIP being capable of absorbing multiple cross-reactants.

9. The method according to claim 1 where said liquid sample is contacted with said MIP by adding said liquid sample to a column that is packed with said MIP.

10. The method according to claim 9 where said liquid sample in said column is separated from said column by applying pressure to said column via a pumping mechanism and collecting the sample liquid emerging from said column.

11. The method according to claim 1 where said binding assay is an enzyme assay.

12. The method according to claim 11 where said enzyme assay is an enzyme linked immunosorbent assay (ELISA).

13. The method according to claim 11 where said enzyme assay is a homogeneous enzyme immunoassay.

14. The method according to claim 1 where said separated liquid sample is aliquoted into multiple vessels and multiple binding assays are performed on said aliquited liquid samples in said multiple vessels for multiple target analytes, whereby a single binding assay for a target analyte is performed in each of said multiple vessels.

15. The method according to claim 1 where multiplexed binding assays are performed on said separated liquid sample for multiple target analytes.

16. A lateral flow binding assay device comprising:
a) a sample addition pad that is in direct contact with a proximal end of a porous strip;
b) a first zone within said porous strip that is adjacent to said proximal end of said porous strip and contains an MIP that is templated to absorb one or more of said cross-reactants;
c) a second zone located downstream of said first zone within said porous strip comprising one or more localized regions of analyte or receptors bound to said strip, whereby the presence of an analyte can be visually determined following a binding reaction between an analyte within said sample and a reagent; and
d) an absorbing material located at a distal end of said porous strip.

17. The device according to claim 16 wherein said MIP is made from a mixture of several individual MIPs that are each individually cast for a separate cross-reactant prior to being mixed together, the resulting mixture being capable of absorbing multiple cross-reactants.

18. The device according to claim 16 wherein said MIP is a multi-receptor MIP in which casts for multiple analytes are incorporated into the MIP, the resulting MIP being capable of absorbing multiple cross-reactants.

19. The device according to claim 16 wherein said sample addition pad contains dried reagents.

20. The device according to claim 16 wherein dried reagents are incorporated into a third zone located between said first zone and said second zone of said porous strip.

21. The device according to claim 16 wherein said binding assay is a competitive assay.
